(19) United States
(12) Patent Application Publication
(10) Pub. No.: US 2011/0059167 A1
(43) Pub. Date: Mar. 10, 2011

(54) ENCAPSULATION OF BIOLOGICALLY ACTIVE AGENTS

(75) Inventors: Mehmet Fidanboylu, London (GB); Irene Papanicolaou, Herfordshire (GB)

(73) Assignee: Glaxo Group Limited

(21) Appl. No.: 12/991,521

(22) PCT Filed: May 5, 2009
(86) PCT No.: PCT/EP2009/055437

§ 371 Date: Nov. 8, 2010

Related U.S. Application Data
(60) Provisional application No. 61/050,775, filed on May 6, 2008.
(60) Provisional application No. 61/074,171, filed on Jun. 20, 2008.

(51) Int. Cl. A61K 9/48; A61K 39/395; A61P 25/28; A61P 35/02; A61P 27/02; A61P 43/00; A61P 31/14; A61P 9/10; A61K 38/02; B29D 22/00

(52) U.S. Cl. 424/455; 424/130.1; 514/1.1; 264/4.6

(57) ABSTRACT

The present invention provides methods of encapsulating biologically active agents such as proteins in particulate carriers such as nanoparticles using Hip agents. Also provided are compositions comprising particulate carriers obtainable by such methods and uses of such compositions in treatment.

### BACKGROUND

A number of drugs have activity at targets in the brain or in the eye, in order to get these to their target they must pass through a biological barrier such as the blood brain barrier. While some molecules are able to cross biological barriers, there are others which do not pass these barriers efficiently or in fact at all. Many drugs are also only efficient when given directly into the target tissue and if this target tissue cannot be reached the drug simply cannot work. Therefore many potentially potent drugs are not useful clinically due to their inability to pass such biological barriers.

A number of approaches have been described in the art to increase drug penetration through these biological barriers.

One approach has been to alter the function of the barrier itself. For instance, osmotic agents or cholinomimetic arecolines result in the opening, or a change in the permeability, of the blood brain barrier (Saija A et al, J Pharm. Pha. 42:135-138 (1990)).

Another approach resides in the modification of the drug molecules themselves. For instance modifications of proteins to attempt passage across the blood brain barrier include glycating such proteins, or alternatively by forming a prodrug. (WO/2006/029845).

Still another approach is the implantation of controlled release polymers which release the active ingredient from a matrix system directly into the nervous tissue. However, this approach is invasive and requires surgical intervention if implanted directly into the brain or spinal cord (sable et al. U.S. Pat. No. 4,833,666) this presents problems with patient compliance and often only allows for localised delivery within the brain with the administered drug usually draining away very quickly. (WO/2006/029845).

To overcome these limitations drug carrier systems have been used however, a major problem in targeted drug delivery is the rapid opsonisation and uptake of injected carriers by the reticuloendothelial system (RES) especially by the macrophages in the liver and spleen.

There remains therefore a need for an efficient and effective means of delivering macromolecules such as proteins to the brain and to the eye. In particular, it would be desirable to find a method of delivery of macromolecules across the blood brain barrier, which would retain activity on entry into the brain, and which may also provide desirable release kinetics, maintain protein stability and activity, and have the ability to evade clearance mechanisms.

### BRIEF DESCRIPTION OF THE FIGURES

FIG. 1 shows the sizing data obtained for a nanoparticle formulation by dynamic light scattering (DLS) that indicate the presence of nanoparticles prepared via the hollow method in suspension.

FIG. 1(a) Correlogram obtained following analysis of a nanoparticle suspension by dynamic light scattering.

FIG. 1(b) Multimodal size distribution (derived data) of the nanoparticles plotted to depict the distribution of the particle population (number) over a range of sizes.

FIG. 1(c) Multimodal size distribution (derived data) of the nanoparticles plotted to depict the distribution of the particle population (number) over a range of sizes.

FIG. 1(d) Multimodal size distribution (derived data) of the nanoparticles plotted to depict the distribution of the particle population (number) over a range of sizes.

FIG. 2.—Nanoparticles analysed by SEM

FIG. 3—Image of hollow nanoparticles by TEM, with a superimposed image of solid PBCA nanoparticles for comparison.

FIG. 4—Encapsulation efficiency measurements of monoclonal IgG1 (anti-CD23).

FIG. 5—Release profile obtained following enzymatic degradation of particles and analysis of the released enzyme by ELISA.

FIG. 6—Determination of the encapsulation efficiency of a domain antibody (hen egg lysozyme dAb) by the bicinchoninic acid assay (BCA assay) in a hollow PBCA nanoparticle.

FIG. 7—Encapsulation efficiency measurements of monoclonal IgG1 (anti-CD23).

### SUMMARY OF INVENTION

In one aspect of the present invention there is provided a method of encapsulating biologically active agents in particulate carriers such as methods of encapsulating proteins and or peptides in, or in and on, or with nanoparticles and a method of delivery of proteins and or peptides across the blood brain barrier by encapsulation in, or in and on, or with nanoparticles and a method of delivery of proteins and or peptides to the eye by encapsulation in, or in and on, or with particulate carriers.

In another embodiment of the present invention there are provided particulate carriers comprising a particle forming substance and a biologically active agent such as a protein and or peptide, for delivery of a protein and or peptide from the blood to the brain across the blood brain barrier or for delivery to the eye. In another embodiment of the invention are compositions of nanoparticles and their use in treating disorders or diseases of the central nervous system and or eye.

### DETAILED DESCRIPTION OF INVENTION

The present invention provides particulate carriers comprising a particle forming substance and a biologically active agent, and methods of making said particulate carriers.

In one embodiment there is provided a polymeric particulate carrier comprising a biologically active agent in an aqueous phase in a hollow lumen.

In another embodiment of the present invention there is provided a method of encapsulating biologically active agents in particulate carriers for ocular delivery comprising the steps of:
a) dissolving a polymer in an organic solvent to form a polymer solution;
b) adding an aqueous solution containing a biologically active agent to the polymer solution to form a primary emulsion of aqueous phase droplets in a continuous organic phase;
c) mixing the primary emulsion with an aqueous medium to form a W/O/W emulsion; and
d) allowing the organic phase to evaporate and thereby obtain particulate carriers comprising a hollow lumen containing said biologically active agent in an aqueous phase.

In a further embodiment the ocular delivery is periocular, for example trans-scleral, subconjunctival, sub-tenon, peribulbar, topical, retrobulbar or is delivered to the inferior, superior or lateral rectus muscle. In one embodiment the ocular delivery is trans-scleral.

Allowing the organic phase to evaporate may be passive or active. For example active evaporation may be by the use of heat.

In another embodiment of the present invention there is provided a method of producing nanoparticles for delivery of proteins to the blood brain barrier comprising the steps of:
a) dissolving a polymer in an organic solvent to form a polymer solution;
b) adding an aqueous solution containing protein to the polymer solution to form a primary emulsion of aqueous phase droplets in a continuous organic phase;
c) mixing the primary emulsion with an aqueous medium to form a W/O/W emulsion; and
d) allowing the organic phase to evaporate and thereby obtain nanoparticles comprising a hollow lumen containing said proteins in an aqueous phase.

Allowing the organic phase to evaporate may be passive or active. For example active evaporation may be by the use of heat.

In a further embodiment the polymer used in any of the methods as described above is selected from but not limited to: poly-L-lactide (PLA), poly(lacto-co-glycolide) (PLG), poly(lactide), poly(caprolactone), poly(hydroxybutyrate) and/or copolymers thereof. Suitable particle-forming materials include, but are not limited to, poly(dienes) such as poly(butadiene) and the like; poly(alkenes) such as polyethylene, polypropylene, and the like; poly(acrylics) such as poly(acrylic acid) and the like; poly(methacrylics) such as poly(methyl methacrylate), poly(hydroxyethyl methacrylate), and the like; poly(vinyl ethers); poly(vinyl alcohols); poly(vinyl ketones); poly(vinylhalides) such as poly(vinyl chloride) and the like; poly(vinyl nitriles), poly(vinyl esters) such as poly(vinyl acetate) and the like; poly(vinyl pyridines) such as poly(2-vinyl pyridine), poly(5-methyl-2-vinyl pyridine) and the like; poly(styrenes); poly(carbonates); poly(esters); poly(orthoesters); poly(esteramides); poly(anhydrides); poly(urethanes); poly(amides); cellulose ethers such as methyl cellulose, hydroxyethyl cellulose, hydroxypropyl methyl cellulose, and the like; cellulose esters such as cellulose acetate, cellulose acetate phthalate, cellulose acetate butyrate, and the like; poly(saccharides), proteins, gelatin, starch, gums, resins, and the like. These materials may be used alone, as physical mixtures (blends), or as copolymers. Also polyacrylates, polymethacrylates, polybutylcyanoacrylates, polyalkylcyanoacrylates, polyarylamides, polyanhydrates, polyorthoesters, N,N-L-lysinediylterephthalate, polyanhydrates, desolvated biologically active agents or carbohydrates, polysaccharides, polyacrolein, polyglutaraldehydes and derivatives, copolymers and polymer blends.

In another embodiment of the present invention there is provided a method of encapsulating biologically active agents by producing particulate carriers comprising the steps of:
a) dissolving polybutylcyanoacrylate (PBCA) in an organic solvent to form a polymer solution;
b) adding an aqueous solution containing a biologically active agent to the polymer solution to form a primary emulsion of aqueous phase droplets in a continuous organic phase;
c) mixing the primary emulsion with an aqueous medium to form a W/O/W emulsion; and
d) allowing the organic phase to evaporate and thereby obtain particulate carriers comprising a hollow lumen containing said biologically active agent in an aqueous phase.

Allowing the organic phase to evaporate may be passive or active. For example active evaporation may be by the use of heat.

In a further embodiment of the method s as herein described, step (d) additionally comprises the addition of gel forming polymers. In a further embodiment the gel forming polymer is agarose.

In one embodiment the particulate carriers of the present invention comprise biologically active agents such as proteins or peptides. Such proteins may be antigen binding molecules which as used herein refers to antibodies, antibody fragments and other protein constructs which are capable of binding to a target.

Antigen binding molecules may comprise a domain. A “domain” is a folded protein structure which has tertiary structure independent of the rest of the protein.

Generally, domains are responsible for discrete functional properties of proteins and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

Antigen binding molecules may comprise at least one immunoglobulin variable domain, for example such molecules may comprise an antibody, a domain antibody, Fab, Fab′, F(ab′)₂, Fv, ScFv, diabody, heteroconjugate antibody. Such antigen binding molecules may be capable of binding to a single target, or may be multispecific, i.e. bind to a number of targets, for example they may be bispecific or trispecfic. In one embodiment the antigen binding molecule is an antibody. In another embodiment the antigen binding molecule is a domain antibody (dAb). In yet a further embodiment the antigen binding molecule may be a combination of antibodies and antigen binding fragments such as for example, one or more dAbs and or one or more ScFvs attached to a monoclonal antibody. In yet a further embodiment the antigen binding molecule may be a combination of antibodies and peptides. Antigen binding molecules may comprise at least one non-Ig binding domain such as a domain that specifically binds an antigen or epitope independently of a different V region or domain, this may be a dAb, for example a human, camelid or shark immunoglobulin single variable domain or it may be a domain which is a derivative of a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEl and GroES; transferrin (trans-body); ankyrin repeat protein (DARPin); peptide aptamer; C-type lectin domain (Tetranectin); human-crystallin and human ubiquitin (affilins); PDZ domains; scorpion toxinkunitz type domains of human protease inhibitors; and fibronectin (adnectin); which has been subjected to protein engineering in order to obtain binding to a ligand other than the natural ligand.

CTLA-4 (Cytotoxic T Lymphocyte-associated Antigen 4) is a CD28-family receptor expressed on mainly CD4+ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies. For further details see Journal of Immunological Methods 248 (1-2), 31-45 (2001)

Lipocalins are a family of extracellular proteins which transport small hydrophobic molecules such as steroids, bilins, retinoids and lipids. They have a rigid-sheet secondary structure with a numer of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633

An affibody is a scaffold derived from Protein A of Staphylococcus aureus which can be engineered to bind to antigen. The domain consists of a three-helical bundle of approximately 58 amino acids. Libraries have been generated by randomisation of surface residues. For further details see Protein Eng. Des. Sel. 17, 455-462 (2004) and EP1641818A1

Avimers are multidomain proteins derived from the A-domain scaffold family. The native domains of approximately 35 amino acids adopt a defined disulphide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007)

A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999).

Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two-helices and a-turn. They can be engineered to bind different target antigens by randomising residues in the first-helix and a-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1.

Fibronectin is a scaffold which can be engineered to bind to antigen. Adnectins consists of a backbone of the natural amino acid sequence of the 10th domain of the repeating units of human fibronectin type III (FN3). Three loops at one end of the sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1.

Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005).

Microbodies are derived from naturally occurring microproteins of 25-50 amino acids in length which contain 3-4 cysteine bridges—examples of microproteins include KalataB1 and conotoxin and knottins. The microproteins have a loop which can be engineered to include up to 25 amino acids without affecting the overall fold of the microprotein. For further details of engineered knottin domains, see WO2008098796.

Other non Ig binding domains include proteins which have been used as a scaffold to engineer different target antigen binding properties include human-crystallin and human ubiquitin (affilins), kunitz type domains of human protease inhibitors, PDZ-domains of the Ras-binding protein AF-6, scorpion toxins (charybdotoxin), C-type lectin domain (tetranectins) are reviewed in Chapter 7—Non-Antibody Scaffolds from Handbook of Therapeutic Antibodies (2007, edited by Stefan Dubel) and Protein Science 15:14-27 (2006). Non Ig binding domains of the present invention could be derived from any of these alternative protein domains.

In a further embodiment of the invention the antigen binding molecule binds to a target found in the central nervous system such as for example in the brain or spinal cord, or for example in neuronal tissue.

In yet a further embodiment of the invention described herein the antigen binding molecule specifically binds to a target known to be linked to neurological diseases or disorders such as for example MAG (myelin associated glycoprotein), NOGO (neurite outgrowth inhibitory protein) or β-amyloid.

Such antigen binding molecules include antigen binding molecules capable of binding to NOGO for example anti-NOGO antibodies. One example of an anti-NOGO antibody for use in the present invention is the antibody defined by the heavy chain of SEQ ID NO 1 and the light chain of SEQ ID NO 2 or an anti-NOGO antibody or antigen binding fragment thereof which comprises the CDRs of the antibody set out in SEQ ID NO 1 and 2. Further details of this antibody (H28 L16) can be found in PCT application WO2007068750 which is herein incorporated by reference.

Such antigen binding molecules include antigen binding molecules capable of binding to MAG for example anti-MAG antibodies. One example of the anti-MAG antibody for use in the present invention is the antibody defined by the heavy chain variable region of SEQ ID NO 11 and the light chain variable region of SEQ ID NO 12 or an anti-MAG antibody or antigen binding fragment thereof which comprises the CDRs of the antibody set out in SEQ ID NO 1 and 2. Further details of this antibody (BvH1 CvL1) can be found in PCT application WO2004014953 which is herein incorporated by reference.

Such antigen binding molecules include antigen binding molecules capable of binding to β-amyloid for example anti-β-amyloid antibodies. One example of the anti-β-amyloid antibody for use in the present invention is the antibody defined by the heavy chain of SEQ ID NO 5 and or the light chain of SEQ ID NO 6 or an anti-β-amyloid antibody or antigen binding fragment thereof which comprises the CDRs of the antibody set out in SEQ ID NO 5 and 7. Further details of this antibody (H2L1) can be found in PCT application WO2007113172 which is herein incorporated by reference. An alternative anti-β-amyloid antibody Which is of use in the present invention is the anticody defined by the heavy chain of SEQ ID NO 7 and or the light chain of SEQ ID NO 8 or an anti-β-amyloid antibody or antigen binding fragment thereof which comprises the CDRs of the antibody set out in SEQ ID NO 7 and 8.

The CDR sequences of such antibodies can be determined by the Kabat numbering system (Kabat et al; Sequences of proteins of Immunological Interest NIH, 1987), the Chothia numbering system (Al-Lazikani et al., (1997) JMB 273, 927-948), the contact definition method (MacCallum R. M., and Martin A. C. R. and Thornton J. M, (1996), Journal of Molecular Biology, 262 (5), 732-745) or any other established method for numbering the residues in an antibody and determining CDRs known to the skilled man in the art.

In one embodiment of the invention the antigen binding protein binds to a target found in the eye such as for example TNF, TNFr-1, TNFr-2, TGFbeta receptor-2, VEGF, NOGO, MAG, IL-1, IL-2, IL-6, IL-8, IL-17, CD20, Beta amyloid, FGF-2, IGF-1, PEDF, PDGF or a complement factor for example C3, C5, C5aR, CFD, CFH, CFB, CFI, sCR1 or C3,

In one embodiment of the invention the antigen binding protein binds to VEGF. In an alternative embodiment of the invention the antigen binding protein binds to β-amyloid.

In one embodiment of the present invention the particulate carriers may be microspheres or nanoparticles. In one such embodiment the particulate carrier is a nanoparticle and the biologically active agent is a protein. In another embodiment the particulate carrier is a nanoparticle and the biologically active agent is a peptide. In a further embodiment the particulate carrier is a nanoparticle and the biologically active agent comprises an antigen binding molecule for example a domain antibody or antibody. In yet a further embodiment the particulate carrier is a nanoparticle and the biologically active agent comprises a domain. In another embodiment the particulate carrier is a microsphere and the biologically active agent is a protein. In a further embodiment the particulate carrier is a microsphere and the biologically active agent is a peptide. In yet a further embodiment the particulate carrier is a microsphere and the biologically active agent comprises an antigen binding molecule for example a domain antibody or antibody. In yet a further embodiment the particulate carrier is a microsphere and the biologically active agent comprises a domain.

In one embodiment of the present invention there is provided a composition comprising nanoparticles according to any method of the invention as presented herein. In a further embodiment at least about 90% of the nanoparticles by number are within the range of about 1 nm to about 1000 nm when measured using dynamic light scattering techniques. In a further embodiment at least about 90% of the nanoparticles by number are within the range of about 1 nm to about 400 nm, or about 1 nm to about 250 nm or about 1 nm to about 150 nm, or about 40 nm to about 250 nm, or about 40 nm to about 150 nm, or about 40 nm to about 100 nm when measured using dynamic light scattering techniques.

In yet a further embodiment of the present invention at least about 90% of the nanoparticles by number are within the range of about 40 nm to about 250 nm when measured using dynamic light scattering techniques.

In yet a further embodiment of the present invention at least about 90% of the nanoparticles by number are within the range of about 40 nm to about 150 nm when measured using dynamic light scattering techniques.

In yet a further embodiment there is provided a composition comprising the nanoparticles of the present invention wherein the median size of the nanoparticles in the composition is less than about 1000 nm in diameter, for example is less than about 400 nm in diameter for example is less than about 250 nm in diameter, for example is less than about 150 nm in diameter when measured by light scattering techniques.

In yet a further embodiment the median size of the nanoparticles in the composition is about 40 nm to about 250 nm.

In yet a further embodiment the median size of the nanoparticles in the composition is about 40 nm to about 150 nm.

In one embodiment of the present invention there is provided a composition comprising microspheres according to any method of the invention as presented herein. In a further embodiment at least about 90% of the microspheres by number have a diameter within the range of about 1 μm to about 100 μm when measured using Low angle laser light scattering techniques. In a further embodiment at least about 90% of the particles by number are within the range of about 1 μm to about 80 μm, or about 1 μm to about 60 μm or about 1 μm to about 40 μm, or about 1 μm to about 30 μm or about 1 μm to about 10 μm when measured using Low angle laser light scattering techniques.

In yet a further embodiment of the present invention at least about 90% of the microspheres by number are within the range of about 1 μm to about 60 μm when measured using Low angle laser light scattering techniques.

In yet a further embodiment of the present invention at least about 90% of the microspheres by number are within the range of about 1 μm to about 30 μm when measured using Low angle laser light scattering techniques.

In yet a further embodiment there is provided a composition comprising the microspheres of the present invention wherein the median size of the microspheres in the composition is less than about 100 μm in diameter, for example is less than about 80 μm in diameter for example is less than about 60 μm in diameter, for example is less than about 40 μm in diameter when measured by Low angle laser light scattering techniques.

In yet a further embodiment the median size of the microspheres in the composition is about 1 μm to about 6 μm, or 1 μm to about 30 μm.

In another embodiment of the invention the particulate carriers continue to release therapeutic amounts of active biological molecules over a period of at least 3 months or longer, or of up to 6 months or longer or of up to 12 months or longer.

In one embodiment the w/w ratio of protein to polymer may be 0.5% to 50% for example is at least about 0.5% or is at least about 1% or is at least about 2% or is at least about 5% or is at least about 7% or is at least about 10% or is at least about 11% or is at least about 14% or is at least about 20% or is at least about 40%, or is at least about 50%. For example when the protein is a peptide the peptide to polymer ratio may be at least about 11, or when the protein is an antibody the antibody to polymer ratio may be at least about 14%, or when the protein is a domain antibody the domain antibody to polymer ratio may be at least about 11%.

In one embodiment of the present invention the encapsulation efficiency of the particles is at least about 1% or is at least about 2% or is at least about 10% or is at least about 20% or is at least about 40% or is at least about 50% or is at least about 60% or is at least about 70% or is at least about 80% or is at least about 90% or is alt least about 95% or is least about 97% or is at least about 99%. For example when the protein is a peptide the encapsulation efficiency may be at least about 60%, when the protein is an antibody the encapsulation efficiency may be at least about 90%, or when the protein is a domain antibody the encapsulation efficiency may be at least about 60%.

Examples of organic solvents suitable for use with the methods of the invention include but are not limited to water-immiscible esters such as ethyl acetate, isopropyl acetate, n-propyl acetate, isobutyl acetate, n-butyl acetate, isobutyl isobutyrate, 2-ethylhexyl acetate, ethylene glycol diacetate; water-immiscible ketones such as methyl ethyl ketone, methyl isobutyl ketone, methyl isoamyl ketone, methyl n-amyl ketone, diisobutyl ketone; water-immiscible aldehydes such as acetaldehyde, n-butyraldehyde, crotonaldehyde, 2-ethylhexyldehyde, isobutylaldehyde and propionaldehyde; water-immiscible ether esters such as ethyl 3-ethoxypropionate; water-immiscible aromatic hydrocarbons such as toluene xylene and benzene; water-immiscible halohydrocarbons such as 1,1,1 trichloroethane; water-immiscible glycol ether esters such as propylene glycol monomethyl ether acetate, ethylene glycol monoethyl ether acetate, ethylene glycol monobutyl ether acetate, diethylene glycol monobutyl ether acetate; water-immiscible phthalate plasticisers such as dibutyl phthalate, diethyl phthalate, dimethyl phthalate, dioctyl phthalate, dioctyl terephthalate, butyl octyl phthalate, butyl benzyl phthalate, alkyl benzyl phthalate; water-immiscible plasticisers such as dioctyl adipate, triethylene glycol di-2-ethylhexanoate, trioctyl trimellitate, glyceryl triacetate, glyceryl/tripropionin, 2,2,4-trimethyl-1,3-pentanediol diisobutyrate, methylene chloride, ethylacetate or dimethylsulfoxide, carbon tetrachloride, chloroform, cyclohexane, 1,2-dichloroethane, dichloromethane, diethyl ether, dimethyl formamide, heptane, hexane and other hydrocarbons, methyl-tert-butyl ether, pentane, toluene, 2,2,4-trimethylpentane, 1-octanol and its isomers or benzyl alcohol.

In one embodiment of the invention the solvent used in the methods of the invention will be selected from methylene chloride, ethylacetate or dimethylsulfoxide, carbon tetrachloride, chloroform, cyclohexane, 1,2-dichloroethane, dichloromethane, diethyl ether, dimethyl formamide, heptane, hexane and other hydrocarbons, methyl-tert-butyl ether, pentane, toluene, 2,2,4-trimethylpentane, 1-octanol and its isomers, benzyl alcohol.

The particulate carriers, compositions comprising them or methods of making them in all aspects of the present invention as herein described may further comprise the addition of a surfactant such as but not limited to: sodium cholate, poloxamer 188 (pluronic F68™, or F127), polyvinyl alcohol, polyvinyl pyrrolidone, polysorbate 80, dextrans. poloxamers, poloxamines, carboxylic acid esters of multifunctional alcohols, alkoxylated ethers, alkoxylated esters, alkoxylated mono-, di and triglycerides, alkoxylated phenols and diphenols, ethoxylated ethers, ethoxylated esters, ethoxylated triglycerides, substances of the GenapolR™ and BaukiR™ series, metal salts of fatty acids, metal salts of carboxylic acids, metal salts of alcohol sulfates, and metal salts of fatty alcohol sulfates and metal salts of sulfosuccinates and mixtures of two or more of said substances.

In a further embodiment the surfactant is selected from sodium cholate, poloxamer 188 (pluronic F68™), polyvinyl alcohol, polyvinyl pyrrolidone, polysorbate 80 and dextrans.

In one embodiment of the present invention there is provided particulate carriers comprising biologically active agents, obtainable by any of the methods of the invention herein described.

The biologically active agent encapsulated in particulate carriers and or compositions of the present invention retains at least some biological activity on its release from the particulate carrier, for example, a proportion of the molecules in the composition may retain at least some ability to bind to their target when the agent is a binding agent and elicit a biological response on the release of the biologically active agent from the particles. Such binding can be measured in a suitable biological binding assay, examples of suitable assays include but are not limited to ELISA or Biacore™. In a further embodiment the composition retains at least 50% of its affinity for the target, or at least 70% or at least 90% of its affinity (Kd) for the target when measured by a biological binding assay on release from the particles for example in one embodiment as determined by ELISA, Biacore. In one embodiment the composition will be capable of eliciting a therapeutic effect in the subject to which it is administered. The biological activity of the compositions of the invention can be measured by any suitable assay which measures activity of the encapsulated biologically active molecule.

In one embodiment of the present invention there is provided a method of delivering a protein across a biological barrier such as the blood brain barrier by encapsulation of the protein in a nanoparticle to a patient. In a further embodiment the patient is human.

In one embodiment of the present invention there is provided a method of delivering a protein encapsulated in a particulate carrier such as a microsphere to the eye of a mammal, for example a human.

In another embodiment there is provided a pharmaceutical composition comprising a biologically active agent encapsulated in a particulate carrier of the present invention as herein described.

In a further embodiment there is provided a pharmaceutical composition comprising a protein encapsulated in the nanoparticles of the present invention as herein described. In a further embodiment there is provided a pharmaceutical composition comprising a protein encapsulated in microspheres for ocular delivery as herein described for treating and or preventing a disease of the eye.

In a further embodiment a composition of the present invention may be used to treat and or prevent disorders or diseases which involve the particulate carriers crossing the blood brain barrier.

In a further embodiment a composition of the invention as herein described may be used to treat and or prevent disorders or diseases of the Central nervous system, for example it may be used to treat and or prevent Alzheimer's disease, Huntington's disease, bovine spongiform encephalopathy, West Nile virus encephalitis, Neuro-AIDS, brain injury, spinal cord injury, metastatic cancer of the brain, or multiple sclerosis, stroke.

In a further embodiment the composition may comprise an anti-MAG antibody for the treatment and or prevention of stroke or neuronal injury.

In another embodiment the composition may comprise an anti-NOGO antibody for the treatment and or prevention of stroke or neuronal injury or for example for the treatment or prophylaxis of neurodegenerative diseases such as Alzheimer's disease.

In another embodiment the composition may comprise an anti-βamyloid antibody for the treatment and or prevention of stroke or neuronal injury or for example for the treatment or prophylaxis of neurodegenerative diseases such as Alzheimer's disease.

In one embodiment of the invention as herein described the particulate carriers may be administered to the patient by parenteral injection or infusion, intravenous, or intraarterial administration.

In a further embodiment the compositions of the invention as herein described may be used to treat and or prevent disorders or diseases of the eye. In a further embodiment a composition of the invention as herein described may be used to treat and or prevent disorders such as but not limited to age related macular degeneration (neovascular/wet), diabetic retinopathy, retinal venous occlusive disease, uveitis, corneal neovascularisation or glaucoma.

In yet a further embodiment the composition is used to treat and or prevent AMD (age related macular degeneration), for example wet AMD, or dry AMD.

In another embodiment of the present invention there is provided biologically active agents encapsulated in nanoparticles and or microspheres as described herein for use in medicine.

In one embodiment of the present invention there is provided the use of compositions of the invention as described herein in the manufacture of a medicament for the treatment and or prevention of a disease of the central nervous system. In yet another embodiment there is provided the use of a composition of the invention as described herein in the manufacture of a medicament for the treatment and or prevention of Alzheimer's disease. In yet a further embodiment there is provided the use of a composition of the invention as described herein in the manufacture of a medicament for the treatment and or prevention of stroke or neuronal injury.

In another embodiment of the invention there is provided the use of a composition of the invention as described herein in the manufacture of a medicament for the treatment or prevention of ocular diseases such as for example in the manufacture of a medicament for the treatment and or prevention of AMD.

The invention provides methods of treating and or preventing a disease of the central nervous system using a composition of the present invention. In a further embodiment there is provided a method of treating Alzheimer's disease using a composition of the present invention. In yet another embodiment of the present invention there is provided a method of treating and or preventing stroke or neuronal injury using a composition of the present invention.

The invention also provides methods of treating and or preventing ocular disease using a composition of the present invention. In a further embodiment there is provided a method of treating and or preventing AMD using a composition of the present invention.

### DEFINITIONS

As used herein the term “particle forming substance” is used to describe any monomer and or oligomer capable of polymerising, or a polymer which can form an insoluble particle in an aqueous environment for example PBCA, PLGA. The particle forming substance will be soluble in an organic solvent when not polymerised.

The term “particulate carrier” as used throughout this specification is used to cover both nanoparticles and microspheres. “Microspheres” are particles composed of various natural and synthetic materials with diameters larger than 1 μm whereas “nanoparticles” as used herein are submicron sized particles such as for example 1-1000 nm.

In one embodiment the terms particulate carrier, nanoparticles and microspheres as used herein denotes a carrier structure which is biocompatible and sufficiently resistant to chemical and/or physical destruction by the environment of use such that a sufficient amount of the particles remain substantially intact after entry in to the human or animal body following administration and for sufficient time so as to be able to reach the desired target organ or tissue e.g. the brain or the eye.

The term “Biologically active agent” as used herein is a term used to indicate that the molecule must be capable of at least some biological activity when reaching their desired target. For the avoidance of doubt the term “Biologically active agent” and the “biologically active molecule” as used throughout the specification are intended as to have the same meaning and able to be used interchangeably.

The term “solubilisation” is defined as either formation of a solution, in the form of individual molecules in the solvent, or formation of a solid in liquid suspension, in the form of fine solid aggregates of molecules suspended in the liquid. The solubilisation process may also result in a mixture of fully dissolved molecules and suspended solid aggregates.

The term “protein” as used throughout this specification for encapsulation in particulate carriers includes proteins having a molecular weight of at least 11 kDa, or at least 12 kDa, or at least 50 kDa, or at least 100 kDa, or at least 150 kDa or at least 200 kDa. Proteins for encapsulation may also be of considerable length such as at least 70 amino acids in length or at least 100 amino acids in length or at least 150 amino acids in length or at least 200 amino acids in length.

The term “peptide” as used throughout this specification for encapsulation in particulate carriers includes shorter sequences of amino acids having a molecular weight of no more than about 10 kDa, or no more than about 8 kDa, or no more than about 5 kDa, or no more than about 2 kDa or no more than about 1 kDa or is less than 1 Kda. Peptides for encapsulation are no more than 70 amino acids in length or are no more than 50 amino acids in length, or are no more than are no more than 40 amino acids in length, or are no more than 20 amino acids in length or are less than 10 amino acids in length.

The term “Peri-ocular” refers to local administration to positions surrounding the outside of the eye and includes but is not limited to:

“Sub-conjuctival”—underneath the conjuctiva—a clear mucus membrane that covers the eyeball over the sclera; “Sub-tenon”—underneath the Tenon's membrane that envelopes the eye but outside of the sclera; “peribulbar”—the space underneath the globe of the eye where it sits in the eye socket; “retrobulbar”—the space at the very back of the globe of the eye, close to the optic nerve; “supra-choroidal”—underneath the sclera but outside of the choroid into the supra-choroidal space; “trans-scleral”—this term can also be used to mean delivery across, i.e. from outside of the sclera.

The phrase “immunoglobulin single variable domain” refers to an antibody variable domain (V_{H}, V_{HH}, V_{L}) that specifically binds an antigen or epitope independently of a different V region or domain. An immunoglobulin single variable domain can be present in a format (e.g., homo- or hetero-multimer) with other, different variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” which is capable of binding to an antigen as the term is used herein. An immunoglobulin single variable domain may be a human antibody variable domain, but also includes single antibody variable domains from other species such as rodent (for example, as disclosed in WO 00/29004, nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanised according to standard techniques available in the art, and such domains are still considered to be “domain antibodies” according to the invention. As used herein “V_{H} includes camelid V_{HH} domains.

The term “antigen binding molecule” as used herein refers to antibodies, antibody fragments and other protein constructs which are capable of binding to a target.

A “domain” is a folded protein structure which has tertiary structure independent of the rest of the protein. Generally, domains are responsible for discrete functional properties of proteins and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

The term “Light scattering techniques” as used herein is a means used to determine the size distribution profile of small particles in solution—one example of light scattering technique is dynamic light scattering which may be used to measure nanoparticles and another example of light scattering is static light scattering or low angle light scattering which may be used to measure microspheres.

The term “Dynamic light scattering” (DLS) as used herein is a method which utilises the light scattered by particle dispersions to derive information on the size of the particles. Dynamic light scattering relies on the fact that when in liquid suspension, the Brownian motion of particles is dependent on particle size and that the Brownian motion of the particles produces fluctuations in the intensity of light scattered from a particle sample. The particle diameter is derived by analysing these fluctuations by means of a correlation function. The Stokes-Einstein equation is then applied to yield the mean hydrodynamic diameter of the particles.

A multi-exponential analysis can produce a size distribution, providing insight into the presence of different species inside a sample. DLS is generally accepted for the analysis of nanoparticles.

“Static light scattering” or “Low angle laser light scattering” which are used interchangeably throughout the present specification is sometimes referred to as Laser diffraction. Laser diffraction relies on the fact that the diffraction angle is inversely proportional to particle size. The method utilises the full Mie theory which completely solves the equations for the interaction of light with matter. Laser diffraction can be used for the analysis of nanoparticles and microparticles (0.02 to 2000 micrometers in diameter).

The term “Blood brain barrier” (BBB) as used herein is a membranic structure that acts primarily to protect the brain from chemicals in the blood, while still allowing essential metabolic function. It is composed of cerebral microvascular endothelial cells, which are packed very tightly in brain capillaries. This higher density restricts passage of substances from the bloodstream much more than endothelial cells in capillaries elsewhere in the body.

Throughout this specification the percentage drug loading is defined as the percentage of weight of drug per weight of material used in the particle formulation (polymer weight) w/w.

% drug loading=(weight of drug/weight of material used in the particle formulation)×100%.

Within this specification the invention has been described, with reference to embodiments, in a way which enables a clear and concise specification to be written. It is intended and should be appreciated that embodiments may be variously combined or separated without parting from the invention.

### EXAMPLES

### Example 1

### Polymerisation of BCA (butylcyanoacrylate) Monomer

A rapid polymerisation reaction in organic solvent was used to form the polymer:

BCA monomer (200 μl, Vetbond, 3M) was added to 1 ml absolute ethanol in a 25 ml beaker with slow swirling. The resulting solution was gently mixed until the polymerisation reaction was initiated. The polymerisation reaction resulted in the formation of a white solid dispersion. The mixing of the dispersion was stopped as soon as the reaction mixture became too viscous to agitate.

The ethanol in the reaction mixture was then allowed to evaporate in the fume-hood for at least 1 hour. Following evaporation of the ethanol, a cracked white solid cake was obtained. The solid was collected and used in the nanoparticle preparation process.

### Example 2

### Preparation of Hollow Nanoparticles by the Double Emulsion Method

The PBCA polymer was dissolved in dichloromethane at a concentration of 1% w/v and used to prepare hollow PBCA nanoparticles by emulsification into a double emulsion (water in oil in water, w/o/w) as follows:

(i) Primary emulsion (w/o)

Inner phase (w): 5% sodium cholate (SIGMA) in water or buffer, prepared by mixing:

500 μl water or buffer; and

500 μl sodium cholate (10% w/v stock solution).

The total volume of the inner aqueous phase was 1 ml. The solution was kept on ice until it was time to use it. Each solution was drawn into a 1 ml insulin syringe (Terumo 1 ml, BD microlance needle 19 G 1.5″) prior to use.

Outer (organic) phase (o): PBCA polymer (1% w/v) in dichloromethane (“DCM, Fischer).

The organic phase (PBCA polymer in DCM, 6 ml) was poured into a 10 ml beaker (resting on ice to keep cool) and the probe of the homogeniser was inserted (Ultra-Turrax, T25, 50 ml probe). The solution was covered with parafilm (attached to the beaker and probe) and homogenised at a speed of 24,000 rpm using a rotor stator homogeniser (Ulltra-Turrax T25 basic).

Formation of a Primary Emulsion:

As soon as the homogeniser reached the required speed, the inner aqueous phase was added by injecting inside the solution close to the probe. The resulting emulsion was homogenised for 2 minutes (on ice) and then transferred to a glass syringe (SGE, 25 ml, gas-tight, suitable for organic solvents, P/N 009462 25MDR-LL-GT, Batch # F06-A2190, fitted with a blunt 5 cm 2R2 needle, 0.7 mm ID).

(ii) Secondary Emulsion (w/o/w)

Inner phase (w/o): primary emulsion from homogenisation step described above.

Outer phase (w): sodium cholate (1.25% w/v) in water.

Formation of the Secondary Emulsion:

The primary single emulsion (w/o) was used to form a double emulsion (w/o/w) by addition to a secondary aqueous phase (1.25% w/v sodium cholate) with homogenisation. The sodium cholate solution (1.25% w/v, 30 ml) was transferred to a tall 50 ml beaker (resting on ice to keep emulsion cool) and the probe of a Silverson L4RT homogeniser was inserted (¾ inch probe, high emulsor screen). The solution was covered with parafilm (attached to the beaker and probe) and homogenised at a speed of 8,000 rpm. The primary emulsion was injected into the solution close to the probe as soon as the 8,000 rpm speed was reached. The resulting emulsion was homogenised for 6 minutes.

The double emulsion that was formed was transferred to a short 50 ml beaker and the organic phase allowed to evaporate in the fume hood under constant stirring (IKA magnetic stirrer, setting 4) for 3 hours.

Washing of the Nanoparticles by Centrifugation:

Following removal of the organic solvent, the nanoparticles that were formed were washed once by centrifugation at 16,200 rcf and re-suspended in water (10 ml).

### Example 3

### Confirmation of Nanoparticle Formation by Dynamic Light Scattering

The formation of nanoparticles was confirmed by sizing using quasi-elastic light scattering (QELS), also known as dynamic light scattering (DLS). The particles were analysed using a Brookhaven Instruments corporation particle size analyser (BIC 90 plus) following the standard procedure provided by the manufacturer. The particle suspension was diluted 200× in water and sized using standard sizing parameters (temperature of 25° C., laser beam angle of 90°, laser wavelength of 658 nm). The particles were analysed by performing 10 sizing runs of 1 minute in duration each.

The instrument presented the raw data in the standard form of a correlogram. This depicts the autocorrelation function C (τ) of scattered light intensity from the particles at different time intervals and how the autocorrelation decays with the decay time τ. The decay in the autocorrelation of scattered light is dependent on particle diameter and is more rapid for smaller particles. The instrument derives information on particle size by applying the Stokes-Einstein equation. This yields the mean hydrodynamic diameter of the particles in the sample and further derived data on the particle population.

Dynamic light scattering is very sensitive to the presence of large particles, which even when they represent less than 1% of the sample can significantly influence the measurements. As a result, the mean hydrodynamic diameter that the instrument gives, which is heavily influenced by the large particles in the sample could vary substantially. As a result, differences in size of tens of nanometers between batches can be observed and for this reason it is important to look at the complete data set that the instrument provides, with the correlogram being the most important. By looking at the complete data set, it is possible to accurately characterise a sample as the instrument can easily detect the small particle majority that is present in the sample despite the presence of few large particles. The shape of the correlogram itself provides a very clear indication of whether the particles are small, as well as whether the sample is polydisperse. The baseline index also gives an accurate representation of the quality of the data. All of the data in this document exhibited a baseline index that did not fall below 5, with 10 being the maximum for the highest possible quality of a reading.

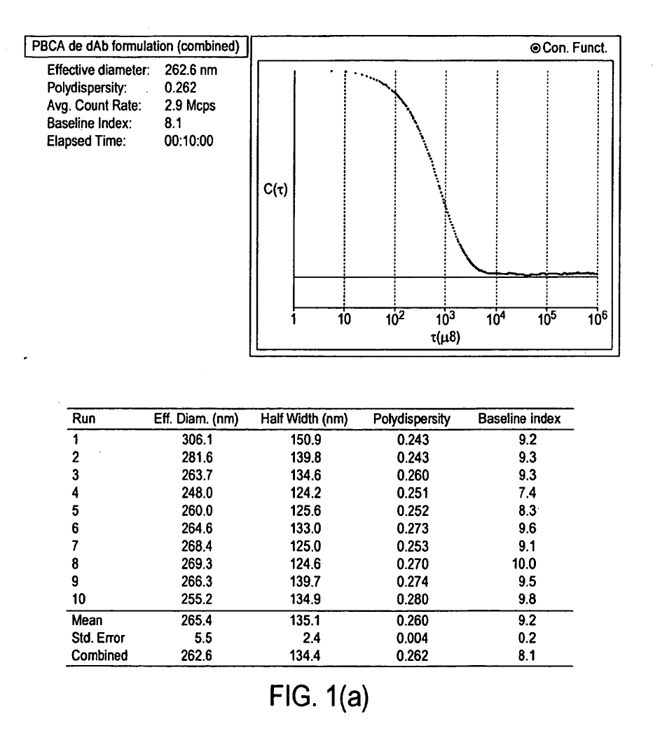

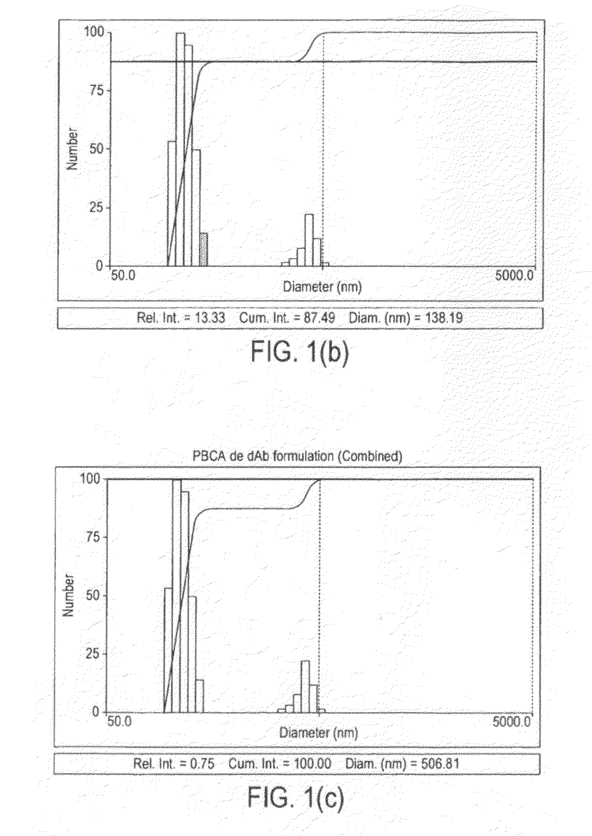

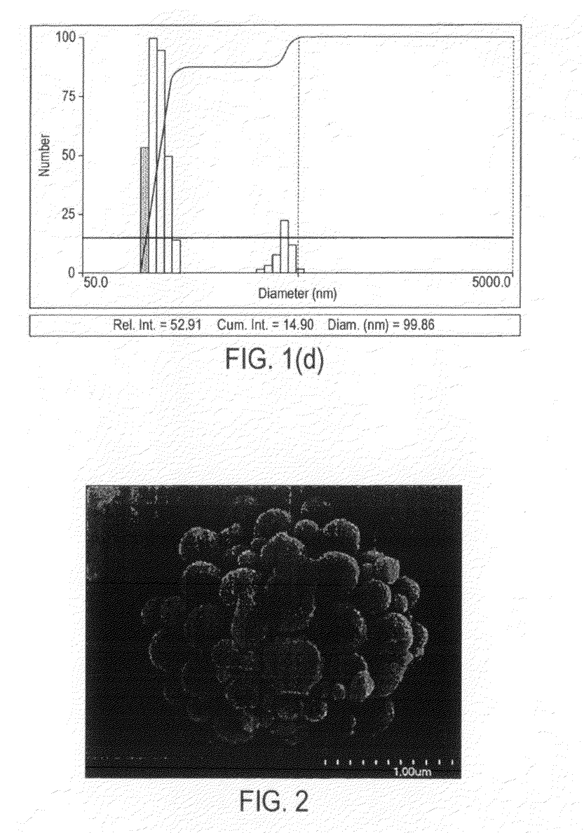

FIG. 1a shows the correlogram (raw data) obtained following sizing of the particle suspension by QELS. The correlogram clearly showed that a nanoparticle suspension had been generated by the particle preparation process, as absence of particles would not generate any light scattering. The shape of the correlogram suggested that the suspension was of good pharmaceutical quality, as the particles were small and no large aggregates were present. Sizing of the nanoparticle suspension by QELS showed that nanoparticles of a mean hydrodynamic diameter of 262.6 nm had formed (FIG. 1a). The particle population was also found to be relatively monodisperse, with the polydispersity index, which is a measure of how broad the range of particle sizes in the sample is, at 0.262 (FIG. 1a). This is below the maximum acceptable value of 0.300 for a particle formulation. In general, the correlogram confirmed that the double emulsion process had successfully generated a good quality suspension of PBCA nanoparticles.

The derived data (generated by the instrument using the Stokes Einstein equation) suggest that the majority of the particles were small (FIGS. 1b-d). The results suggest that approximately 87.5% of the particle population had a diameter of 138.19 nm or lower (FIG. 1b). It was found that the suspension was free of large aggregates and did not contain any particles that exceeded 506.81 nm in diameter, with the majority of the particle population being significantly smaller (FIG. 1c). In addition, the formulation did not contain any particles that were smaller than 99.86 nm (FIG. 1d). Therefore, the majority of the particles were of a diameter between 99.86 and 138.19 nm, a size that is ideal for intravenous administration but not too small so that drug loading is compromised.

FIG. 1.—Sizing data obtained by QELS that indicate the presence of nanoparticles in suspension.

FIG. 1(a) Correlogram obtained following analysis of a nanoparticle suspension by dynamic light scattering. According to the data obtained, the mean hydrodynamic diameter of the particles was 262.6 nm and the polydispersity index 0.262.

FIG. 1(b) Multimodal size distribution (derived data) of the nanoparticles plotted to depict the distribution of the particle population (number) over a range of sizes. The majority (87.5%) of the particle population appeared to have a diameter of 138.19 nm or lower.

FIG. 1(c) Multimodal size distribution (derived data) of the nanoparticles plotted to depict the distribution of the particle population (number) over a range of sizes. The data suggests that 87.5% possessed a diameter of 138.19 nm or lower and that 100% of the particle sample possessed a diameter of 506.81 nm or lower. Therefore, the suspension was free of large aggregates and was therefore considered to be suitable for intravenous administration.

FIG. 1(d) Multimodal size distribution (derived data) of the nanoparticles plotted to depict the distribution of the particle population (number) over a range of sizes. The data suggest that 14.9% of the particle sample possesses a diameter of 99.86 nm or lower.

The process was found to yield similar nanoparticle sizes when different nanoparticle formulations were prepared. Table 1 summarises the sizing data obtained from a series of four different formulation runs:

TABLE 1

| | Mean hydrodynamic | Polydispersity |
|---|---|---|
| Formulation | diameter (nm) | index |
| 1 | 259.6 | 0.164 |
| 2 | 437.6 | 0.281 |
| 3 | 319.2 | 0.303 |
| 4 | 320.6 | 0.248 |
| Average | 334.25 | 0.249 |

In general, the hollow PBCA nanoparticle preparation process was found to generate nanoparticle suspensions that were of the desired diameter and polydispersity.

### Example 4

### Analysis of Nanoparticles—Confirmation of Nanoparticle Formation and Hollow Morphology by Electron Microscopy

In order to confirm that the particles were formed and that they were hollow, samples were visualised by electron microscopy. Nanoparticle suspensions were examined by transmission electron microscopy (TEM). Freeze-dried nanoparticles were analysed by scanning electron microscopy (SEM). Analysis by both microscopy techniques confirmed the formation of nanoparticles. SEM showed that stable nanoparticles were formed. TEM confirmed that the nanoparticles were hollow, possessing an aqueous core surrounded by a PBCA polymer wall.

FIG. 2 shows nanoparticles analysed by SEM

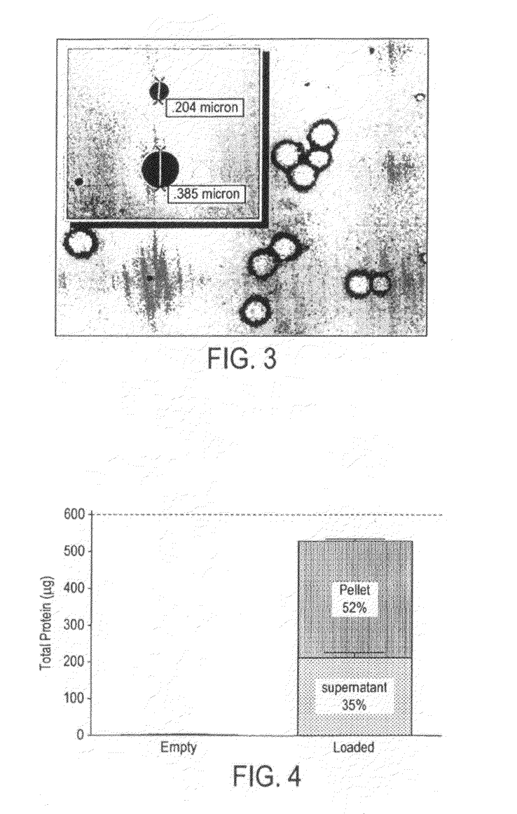

FIG. 3 shows an image of hollow nanoparticles by TEM, with a superimposed image of solid PBCA nanoparticles for comparison.

### Example—5

### Encapsulation of Monoclonal Antibody (Human Anti-CD23) within the Hollow PBCA Nanoparticles

Monoclonal antibody (human anti-CD 23 mAb as disclosed in WO99/58679) was entrapped within the aqueous core of the nanoparticles by inclusion into the inner aqueous phase of the homogenisation process. A solution of the antibody was used to prepare the primary emulsion (w/o), which was then homogenised with the secondary aqueous phase to form the double emulsion (w/o/w) as follows:

(iii) Primary Emulsion (w/o)

Inner phase (w): anti-CD23 mAb as disclosed in WO99/58679 (600 μg in 5% sodium cholate (SIGMA), prepared by mixing:

78 μl mAb solution (7.2 mg/ml)

344 μl H2O

500 μl sodium cholate solution (10% w/v stock solution)

The total volume of the inner aqueous phase was 1 ml. The solution was kept on ice until it was time to use it. Each solution was drawn into a 1 ml insulin syringe (Terumo 1 ml, BD microlance needle 19 G 1.5″) prior to use.

Outer phase (o): PBCA polymer (1% w/v) in dichloromethane (Fischer).

The organic phase (PBCA polymer in DCM, 6 ml) was poured into a 10 ml beaker (resting on ice to keep cool) and the probe of the homogeniser was inserted (Ultra-Turrax, T25, 50 ml probe). The solution was covered with parafilm (attached to the beaker and probe) and homogenised at a speed of 24,000 rpm.

Formation of a Primary Emulsion:

As soon as the homogeniser reached the top speed, the inner aqueous phase was added by injecting inside the solution close to the probe. The resulting emulsion was homogenised for 2 minutes (on ice) and then transferred to a glass syringe (SGE, 25 ml, gas-tight, suitable for organic solvents, P/N 009462 25MDR-LL-GT, Batch # F06-A2190, fitted with blunt 5 cm 2R2 needle, 0.7 mm ID).

(iv) Secondary Emulsion (w/o/w)

Inner phase (w/o): primary emulsion from homogenisation step described above.

Outer phase (w): sodium cholate (1.25% w/v) in water.

Formation of the Secondary Emulsion:

The primary, single emulsion (w/o) was used to form a double emulsion (w/o/w) by addition to a secondary aqueous phase (1.25% w/v sodium cholate) with homogenisation. The sodium cholate solution (1.25% w/v, 30 ml) was transferred to a tall 50 ml beaker (resting on ice to keep emulsion cool) and the probe of a Silverson L4RT homogeniser was inserted (¾ inch probe, high emulsor screen). The solution was covered with parafilm (attached to the beaker and probe) and homogenised at a speed of 8,000 rpm. The primary emulsion was injected into the solution close to the probe as soon as the 8,000 rpm speed was reached. The resulting emulsion was homogenised for 6 minutes.

The double emulsion that was formed was transferred to a short 50 ml beaker and the organic phase allowed to evaporate in the fume hood under constant stirring (IKA magnetic stirrer, setting 4) for 3 hours.

Washing of the Nanoparticles by Centrifugation:

The resulting nanoparticles were pelleted by centrifugation to separate free from encapsulated antibody. Both pellet (entrapped antibody) and supernatant (free antibody) were analysed by total protein assay in order to determine the encapsulation efficiency.

The encapsulation efficiency was found to be 52%, when a total amount of 600 μg antibody was used. The efficiency of encapsulation was found to be sufficiently high to permit the delivery of potentially therapeutic amounts of antibody without exceeding the maximum tolerated dose of PBCA polymer (50 mg/kg in the mouse). Moreover, it was later possible to prepare particles containing a different monoclonal antibody, human anti-IL13, which suggests that the method is applicable to any water-soluble biopharmaceutical.

FIG. 4 show the results obtained from the encapsulation efficiency measurements.

### Example 6

### Release of Monoclonal Antibody from the Nanoparticles

In addition to achieving efficient encapsulation of a biopharmaceutical, it was necessary to demonstrate that the material could be released from the particles following administration and that it retained its activity. The release of active antibody from the particles was initially investigated in vitro by degradation of the particles followed by detection of any released antibody by ELISA. In order to release the encapsulated antibody, particles were treated with a butyl esterase (from porcine liver, SIGMA), which has been reported to cleave the butyl ester of the PBCA polymer. During the reaction (Ringers solution, pH 7.0, 37° C.), samples were taken at different time points (0, 1, 2, 3, 4 and 24 h) and analysed for the presence of active antibody by ELISA.

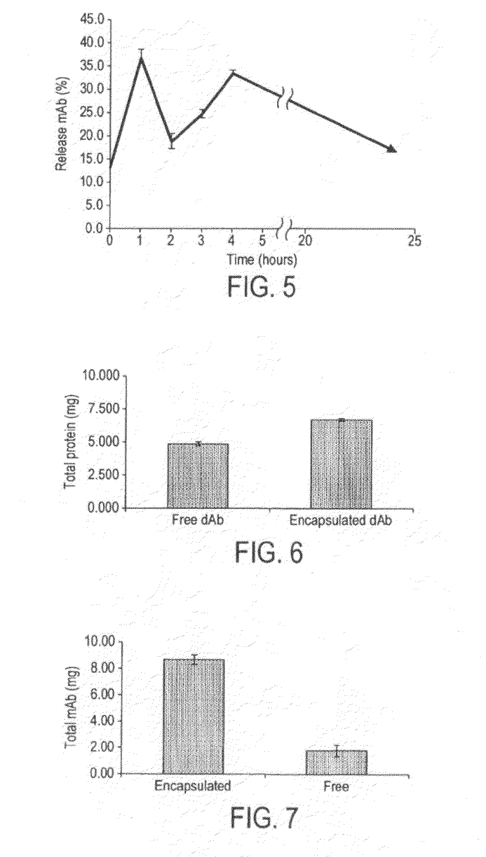

FIG. 5 shows the release profile obtained following enzymatic degradation of the particles and analysis of the released enzyme by ELISA.

### Example 7

### Encapsulation of Domain Antibody (Anti-Hen Egg Lysozyme dAb) within the Hollow PBCA Nanoparticles

In the following examples the BCA assay was performed using a BCA kit obtained from Sigma (QPBCA) and carried out according to the instructions. Free dAb was diluted 2 fold and 10 fold for analysis. The encapsulated dAb was diluted 100 fold.

Domain antibody (anti-hen egg lysozyme dAb) was entrapped within the aqueous core of the nanoparticles by inclusion into the inner aqueous phase of the homogenisation process. In this case, the inner aqueous phase was prepared by mixing 0.5 ml of a 20 mg/ml solution of dAb (10 mg protein) and 0.5 ml of a stabiliser solution (sodium cholate, 10% w/v). The nanoparticles were then prepared by the double emulsion process as described in example 4. The resulting nanoparticles were pelleted by centrifugation to separate free from encapsulated antibody. Both pellet (entrapped antibody) and supernatant (free antibody) were analysed by total protein assay (bicinchoninic acid assay) in order to determine the encapsulation efficiency. The results of the analysis are shown in FIG. 6. The amount of encapsulated dAb was found to be 6.66 mg. The amount of free dAb was 4.83 mg. The efficiency of encapsulation was therefore around 66.6%, with the loading efficiency at 11.1%. It was therefore possible to efficiently encapsulate milligram amounts of protein in the hollow PBCA nanoparticles using the double emulsion method.

### Example 8

### Encapsulation of Monoclonal Antibody (Anti-IL-13 mAb) within the Hollow PBCA Nanoparticles

Monoclonal antibody (anti-IL-13 mAb) was entrapped within the aqueous core of the nanoparticles by inclusion into the inner aqueous phase of the homogenisation process. The inner aqueous phase was prepared by mixing 0.5 ml of a 20 mg/ml solution of mAb (10 mg protein) and 0.5 ml of a stabiliser solution (sodium cholate, 10% w/v). The nanoparticles were then prepared by the double emulsion process as described in Example 5. The resulting nanoparticles were pelleted by centrifugation to separate free from encapsulated antibody. Both pellet (entrapped antibody) and supernatant (free antibody) were analysed by total protein assay (bicinchoninic acid assay) in order to determine the encapsulation efficiency. The results of the analysis are shown in FIG. 12. The amount of encapsulated mAb was found to be 8.62 mg. The amount of free mAb was 1.79 mg. The efficiency of encapsulation was 86.2%, with the loading efficiency 14.4% w/w.

Sequence Listing.

| SEQ ID NO. | Description |
|---|---|
| 1 | Heavy chain amino acid sequence humanised construct H28 |
| | anti-NOGO antibody |
| 2 | 2A10 light chain amino acid sequence humanised construct |
| | L16 anti-NOGO antibody |
| 3 | Heavy chain humanised DNA construct H28 anti-NOGO |
| | antibody |
| 4 | 2A10 light chain humanised DNA construct L16 anti- |
| | NOGO antibody |
| 5 | Mature H2 heavy chain amino acid sequence, (Fc mutated |
| | double mutation bold) beta-amyloid antibody |
| 6 | Mature Light chain amino acid sequence beta-amyloid |
| | antibody |
| 7 | Mature H11 heavy chain amino acid sequence |
| 8 | Mature L9 light chain amino acid sequence |
| 9 | Dalargin hexapeptide |
| 10 | DOM15-26-593 VEGF dAb amino acid sequence |
| 11 | CvL1 variable region amino acid sequence MAG antibody |
| 12 | BvH1 variable region amino acid sequence MAG antibody |
| 13 | H2 Full length DNA beta-amyloid antibody |
| 14 | Optimised L1 light chain DNA beta-amyloid antibody |
| 15 | L1 Full length DNA beta-amyloid antibody |

1. A method of producing a particulate carrier comprising the steps of: a) dissolving polybutylcyanoacrylate (PBCA) in an organic solvent to form a polymer solution; b) adding an aqueous solution containing a biologically active agent to the polymer solution to form a primary emulsion of aqueous phase droplets in a continuous organic phase; c) mixing the primary emulsion with an aqueous medium to form a secondary emulsion; and d) allowing the organic phase to evaporate and thereby obtain particulate carriers comprising a hollow lumen containing the biologically active agent in an aqueous phase.

2. The method of claim 1 wherein step (a) further comprises the addition of a gel forming agent.

3. The method of claim 3 wherein the gel forming agent is agarose.

4. The method of 3 claim 1 wherein the polymer is pegylated.

5. The method of claim 1 wherein the particulate carrier is a microsphere.

6. The method of claim 1 wherein the particulate carrier is a nanoparticle.

7. The method of claim 1 wherein the biologically active agent is a protein or peptide.

8. The method of claim 7 wherein the biologically active agent is an antigen binding molecule.

9. The method of claim 8 wherein the biologically active agent comprises a domain

10. The method of claim 9 wherein the biologically active agent is an antibody.

11. The method of claim 9 wherein the biologically active agent is a domain antibody.

12. A particulate carrier comprising an encapsulated biologically active agent obtainable by the method of claim 1.

13. The particulate carrier of claim 12 wherein the particulate carrier is a nanoparticle.

14. The particulate carrier of claim 13 wherein the biologically active agent is a protein.

15. The particulate carrier of claim 14 wherein the protein to polymer ratio in the nanoparticle is at least about 5% w/w, or is at least about 10% w/w, or is at least about 14% w/w.

16. The particulate carrier of claim 13 wherein the biologically active agent is a peptide.

17. The particulate carrier of claim 16 wherein the peptide to polymer ratio in the nanoparticle is at least about 5% w/w, or is at least about 10% w/w.

18. The particulate carrier of claim 13 wherein the biologically active agent is an antibody.

19. The particulate carrier of claim 18 wherein the antibody to polymer ratio in the nanoparticle is at least about 5% w/w, or is at least about 10% w/w.

20. The particulate carrier of claim 13 wherein the biologically active agent is a dAb.

21. The particulate carrier of claim 20 wherein the dAb to polymer ratio in the nanoparticle is at least about 5% w/w, or is at least about 10% w/w, or is at least about 14% w/w.

22. A pharmaceutical composition comprising the particulate carrier of claim 12.

23. Use of the composition of claim 22 in the treatment or prophylaxis of disease.


SEQUENCE LISTING (15 sequences)

SEQ ID NO: 1 - protein, 462 aa, Artificial Sequence
  Humanised sequence of mus musculus and homo sapiens
MGWSCIILFLVATATGVHSQVQLVQSGAEVKKPGASVKVSCKASGYTFTSYWMHWVRQAPGQGLEWIGNINPSNGGTNYNEKFKSKATMTRDTSTSTAYMELSSLRSEDTAVYYCELMQGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 238 aa, Artificial Sequence
  Humanised sequence of mus musculus and homo sapiens
MGWSCIILFLVATATGVHSDIVMTQSPLSNPVTLGQPVSISCRSSKSLLYKDGKTYLNWFLQRPGQSPQLLIYLMSTRASGVPDRFSGGGSGTDFTLKISRVEAEDVGVYYCQQLVEYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 1389 bp, Artificial Sequence
  Humanised sequence of mus musculus and homo sapiens
atgggatggagctgtatcatcctcttcttggtagcaacagctacaggtgtccactcccaggtgcagctggtgcagtctggggctgaggtgaagaagcctggggcctcagtgaaggtttcctgcaaggcatctggatacaccttcaccagctactggatgcactgggtgcgacaggcccctggacaagggcttgagtggatcggaaatattaatcctagcaatggtggtactaactacaatgagaagttcaagagcaaggccaccatgaccagggacacgtccacgagcacagcctacatggagctgagcagcctgagatctgaggacacggccgtgtattactgtgaactgatgcagggctactggggccagggaacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - DNA, 717 bp, Artificial Sequence
  Humanised sequence of mus musculus and homo sapiens
atgggatggagctgtatcatcctcttcttggtagcaacagctacaggtgtccactccgatattgtgatgacccagtctccactctccaaccccgtcacccttggacagccggtctccatctcctgcaggtctagtaagagtctcctatataaggatgggaagacatacttgaattggtttctccagaggccaggccaatctccacagctcctaatttatttgatgtccacccgtgcatctggggtcccagacagattcagcggcggtgggtcaggcactgatttcacactgaaaatcagcagggtggaggctgaggatgttggggtttattactgccaacaacttgtagagtatccgctcacgtttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggacaacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 5 - protein, 445 aa, Artificial Sequence
  Humanised sequence of mus musculus and homo sapiens
EVQLVESGGGLVQPGGSLRLSCAVSGFTFSDNGMAWVRQAPGKGLEWVSFISNLAYSIDYADTVTGRFTISRDNAKNSLYLQMNSLRAEDTAVYYCVSGTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 219 aa, Artificial Sequence
  Humanised sequence of mus musculus and homo sapiens
DIVMTQSPLSLPVTPGEPASISCRVSQSLLHSNGYTYLHWYLQKPGQSPQLLIYKVSNRFSGVPDRFSGSGSGTDFTLKISRVEAEDVGVYYCSQTRHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 442 aa, Artificial Sequence
  Humanised sequence of mus musculus and homo sapiens
QVQLVQSGAEVKEPGASVKVSCKGSGFNIKVYYVHWLRQLPGKGLEWIGRIDPENGETIYTPKFQDKATLTVDTSTDTAYMELSSLRSEDTAVYYCVSSGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 218 aa, Artificial Sequence
  Humanised sequence of mus musculus and homo sapiens
DIVMTQSPLSNPVTPGEPASISCRSSKSLLHRNGITYLYWYLQKPGQSPQLLIYQMSNLASGVPDRFSSSGSGTDFTLKISRVEAEDVGVYYCAQNLELWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 6 aa, Artificial Sequence
  Humanised sequence of mus musculus and homo sapiens
YAGFLR

SEQ ID NO: 10 - protein, 130 aa, Artificial Sequence
  Humanised sequence of mus musculus and homo sapiens
EVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSSAAAEQKLISEEDLN

SEQ ID NO: 11 - protein, 115 aa, Artificial Sequence
  Humanised sequence of mus musculus and homo sapiens
DIVMTQSPDSLAVSLGERATINCKSSHSVLYSSNQKNYLAWYQQKPGQPPKLLIYWASTRESGVPDRFSGSGSGTDFTLTISSLQAEDVAVYYCHQYLSSLTFGQGTKLEIKRTV

SEQ ID NO: 12 - protein, 126 aa, Artificial Sequence
  Humanised sequence of mus musculus and homo sapiens
QVQLVQSGSELKKPGASVKVSCKASGYTFTNYGMNWVRQAPGQGLEWMGWINTYTGEPTYADDFTGRFVFSLDTSVSTAYLQISSLKAEDTAVYYCARNPINYYGINYEGYVMDYWGQGTLVTVSS

SEQ ID NO: 13 - DNA, 1335 bp, Artificial Sequence
  Humanised sequence of mus musculus and homo sapiens
gaggtgcagctggtggagtctgggggaggcttggtacagcctggggggtccctgagactctcctgtgcagtctctggattcaccttcagtgacaacggaatggcgtgggtccgccaggctccagggaaggggctggagtgggtttcattcattagtaatttggcatatagtatcgactacgcagacactgtgacgggccgattcaccatctccagagacaatgccaagaactcactgtatctgcaaatgaacagcctgagagccgaggacacggctgtgtattactgtgtcagcgggacctggtttgcttactggggccagggcacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 14 - DNA, 657 bp, Artificial Sequence
  Humanised sequence of mus musculus and homo sapiens
gacatcgtgatgacccagagccccctgagcctgcccgtgacccctggcgagcccgccagcatcagctgtagagtgagccagagcctgctgcacagcaacggctacacctacctgcactggtatctgcagaagcctggccagagccctcagctgctgatctacaaggtgtccaaccggttcagcggcgtgcctgatagattcagcggcagcggctccggcaccgacttcaccctgaagatcagcagagtggaggccgaggatgtgggcgtgtactactgctcccagaccagacacgtgccttacacctttggcggcggaacaaaggtggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 15 - DNA, 657 bp, Artificial Sequence
  Humanised sequence of mus musculus and homo sapiens
gatattgtgatgactcagtctccactctccctgcccgtcacccctggagagccggcctccatctcctgcagagttagtcagagccttttacacagtaatggatacacctatttacattggtacctgcagaagccagggcagtctccacagctcctgatctataaagtttccaaccgattttctggggtccctgacaggttcagtggcagtggatcaggcacagattttacactgaaaatcagcagagtggaggctgaggatgttggggtttattactgctctcaaactagacatgttccgtacacgttcggcggagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggacaacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt